(19) United States
(12) Patent Application Publication
(10) Pub. No.: US 2024/0180984 A1
(43) Pub. Date: Jun. 6, 2024

(54) METHODS FOR DETERMINING TUMOR IMMUNE STATUS

(71) Applicant: Duke University, Durham, NC (US)

(72) Inventors: Smita NAIR, Durham, NC (US); Michael BROWN, Durham, NC (US); David Ashley, Durham, NC (US)

(21) Appl. No.: 18/553,703

(22) PCT Filed: Apr. 4, 2022
(86) PCT No.: PCT/US22/23323

§ 371 Date: Oct. 2, 2023

Related U.S. Application Data
(60) Provisional application No. 63/170,097, filed on Apr. 2, 2021.
(60) Provisional application No. 63/179,592, filed on Apr. 26, 2021.
(60) Provisional application No. 63/278,203, filed on Nov. 11, 2021.

(51) Int. Cl. A61K 35/768; A61K 45/06; A61P 35/00; C12N 7/00; G01N 33/569; G01N 33/68

(52) U.S. Cl. CPC A61K 35/768; A61K 45/06; A61P 35/00; C12N 7/00; G01N 33/56983; G01N 33/6866; C12N 2770/32632; G01N 2333/525; G01N 2333/565; G01N 2800/52

(57) ABSTRACT

The present disclosure provides a method of measuring the production of a cytokine in a sample in response to an agonist to determine responsiveness of a patient to immunotherapy. The present disclosure also provides a method of treating a cancer patient with an oncolytic viral therapy when a sample from the patient has a certain level of anti-viral antibodies. Further, the present disclosure describes, in part, an immune competence blood test that allows for one skilled in the art to determine if a tumor is immunoproficient to respond to an immunotherapy.

### CROSS-REFERENCE TO RELATED APPLICATIONS

This patent application claims the benefit of priority of U.S. Provisional Patent Application No. 63/170,097, filed Apr. 2, 2021, U.S. Provisional Patent Application No. 63/179,592, filed Apr. 26, 2021 and U.S. Provisional Patent Application No. 63/278,203, filed Nov. 11, 2021, all of which are incorporated herein by reference in their entirety.

### BACKGROUND

Immune homeostasis, i.e. how the immune system maintains equilibrium and stability, is critical as loss of immune homeostasis impacts our ability to combat diseases. For example, an underactive or failing immune system impacts our ability to combat cancer and infections while an overactive immune system leads to autoimmune disorders. The immune system is broadly divided into two components: innate and adaptive. The induction of systemic adaptive immunity is critical for controlling tumor growth and preventing tumor recurrence. Adaptive immunity is preceded by innate immunity. The innate immune system is activated when pattern-recognition receptors (PRRs), which includes toll-like receptors (TLRs) and non-TLRs, on cells sense molecular patterns associated with pathogens and damaged cells. An inflammatory response is initiated when PRRs on immune cells sense danger through Damage-Associated Molecular Patterns (DAMPs) or pathogens through Pathogen-Associated Molecular Patterns (PAMPs). The process of engaging PRRs through DAMPs and PAMPs leads to acute inflammation, which is critical for 1) innate immune function, 2) transition from innate to adaptive immune responses, and 3) subsequent development of immunological T and B cell memory. Interestingly, this transition from innate to adaptive immune responses (antitumor immune responses in the case of malignancy) is tightly linked with the resolution of inflammation and restoration of immune homeostasis. Increasing evidence suggests that the inability to resolve inflammation leads to chronic inflammation and persistent stimulation of innate immune sensors and ultimately, this disequilibrium leads to immune dysfunction and immunosuppression, an outcome that can promote cancer progression.

Chronic inflammation is prolonged and intensified in the presence of excessive DAMPs that are released from stressed/dying cells within a tumor, a process that is accelerated with standard of care cytotoxic and surgical therapies. Naqvi et al recently demonstrated that pancreatic cancer patients have higher levels of DAMPs in blood than healthy controls, and DAMP levels increase with disease burden and following chemoradiation therapy and surgery. Studies in breast, lung and pancreatic cancer have shown that presence of DAMPs in blood results in tumor cells that are more invasive and aggressive in vitro. Such DAMPs not only maintain a state of systemic chronic inflammation, but this chronic inflammation leads to compromised immune function and inability to respond appropriately to stimuli due to sustained stimulation of PRRs on innate immune cells.

The ability of innate immune cells (monocytes, dendritic cells, neutrophils etc.) to functionally respond to agonists/stimulatory molecules through pattern recognition receptors (PRRs) is called innate immune competence and consequently is a measure of innate immune function. The rationale for using whole blood to study innate immune function is that immune cells that infiltrate into tumors are recruited from the periphery/blood. Cancer growth results in systemic chronic inflammation which compromises immune function/immune dysfunction in the periphery. For example, monocytes in blood are recruited into tumors where they differentiate into inflammatory monocytes or macrophages. Given that immune cells in the tumor represent cells that traffic from blood to the tumor and influence tumor growth through a process called cancer immune-editing, measuring the function of immune cells in blood will predict the immune status of the tumor. This knowledge is critical for application of most ongoing studies that utilize innate immune agonists, for example, oncolytic viruses, STING agonists, and TLR agonists.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present invention is based, in part, on the discovery by the inventors of a novel immune competence blood test that allows for one skilled in the art to determine if cancer growth and/or standard of care cancer therapy compromises innate and adaptive immune function. As shown herein, such assessments of the immune function of a biological sample, such as peripheral blood or tumor biopsy, predicts responsiveness of the tumor to cancer treatment, such as immunotherapy.

Accordingly, the present disclosure provides a method comprising contacting a patient sample with an innate immune agonist, and measuring the level of at least one cytokine, wherein the level of the cytokine is indicative of immune competence and responsiveness of the patient to an immunotherapy. The method may be used to determine the immune proficiency of patient cells, or a tissue therein, to respond to an innate immunity stimulus, immunotherapeutic, or immunotherapy. The innate immune agonist may be selected from the group consisting of oncolytic viruses, STING agonists, TLR agonists and combinations thereof. The sample may be a blood or plasma sample or may include a tumor biopsy. The inflammatory cytokine measured may be selected from TNF-α and IL-12. The method may be used to select subjects diagnosed with cancer for administration of an immunotherapy as those subjects showing immune responsiveness in the sample are more likely to be responsive to immunotherapy including administration of an oncolytic viral therapy.

In another aspect, the present disclosure provides a method of treating a subject with an oncolytic viral therapy, the method comprising: (a) obtaining a serum sample from a subject diagnosed with a cancer; (b) detecting the level of antibody specific to a viral antigen in the serum sample; (c) comparing the level of the antibody in the sample to a reference level; and (d) treating the subject with an oncolytic viral therapy when the level of the antibody in the sample is above the reference level. It has been surprisingly discovered that subjects having high pre-existing neutralizing antibody titers to an oncolytic virus, or related virus thereto, prior to administration of the oncolytic virus or a related booster virus for the treatment of a tumor have significantly improved therapeutic outcomes, including overall survival (OS) improvement.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1 shows peripheral innate immune responsiveness of monocytes correlates with immune activity in the tumor in patients with melanoma in accordance with one embodiment of the present disclosure. Left: Fresh whole blood from patients 1 and 2 was treated with saline, LPS (lipopolysaccharide) or an RNA virus for 4 hours. TNFα and IL-12 production was measured using intracellular cytokine staining in gated monocytes. Patient 1 produced 82% and 81% TNFα in response to LPS and virus, respectively. Patient 2 produced 39% and 34% TNFα in response to LPS and virus, respectively. Right: RNAseq was performed in pretreatment tumors from patients 1 and 2 and analyzed for cell-type specific signatures by ssGSEA (single sample gene set enrichment score).

FIG. 2 is a graph showing the innate immune response in blood correlates with immune response in tumor in accordance with one embodiment of the present disclosure. Blood and tumor samples were collected from 12 patients with pancreatic cancer. Whole blood was either untreated or treated with virus for 4 hours. TNFα production was measured using intracellular cytokine staining in gated monocytes. CXCL10 production was measured in supernatant from tumor tissue slices that were untreated or treated with virus.

FIG. 3 shows an analysis of immune function in response to agonists in accordance with one embodiment of the present disclosure. Patient peripheral blood mononuclear cells (PBMCs) were treated as shown in (a). Cytokine release was measured, and values were divided by baseline (mock) treatment values after TLR1/2, TLR4, and T cell stimulation as shown in (b).

FIG. 4 shows cytokine release by immune cells in response to agonists in accordance with one embodiment of the present disclosure. Induction of cytokine release (fold mock) 24 hours after stimulation with the denoted stimuli comparing African American (AA) vs Caucasian American (CA). P values are from unpaired t-test (two-tailed); Bonferroni correction of 0.004 should be applied.

FIG. 5 shows TLR1/2 responses and survival in patients treated with Provenge (cell-based cancer vaccine) in accordance with one embodiment of the present disclosure. Survival comparison by race (Top left), or comparison by median fold mock induction of each denoted cytokine after TLR1/2 stimulation. P values are from Mantel-Cox Log-Rank test (two-tailed).

FIG. 6 shows TLR1/2 responses and survival in CA and AA patients treated with Provenge in accordance with one embodiment of the present disclosure. Data from FIG. 5, separated by CA or AA race status (left two columns; p values are from Mantel Cox Log rank test); right most panels show comparison between fold mock induction of each cytokine used to separate patients comparing CA vs AA race status (from FIG. 4; p values are from unpaired t-test, two-tailed).

FIG. 7 shows TLR4 responses and survival in patients treated with Provenge in accordance with one embodiment of the present disclosure. Survival comparison by race (Top left), or comparison by median fold mock induction of each cytokine shown after LPS (TLR4) agonist treatment. P value is from Mantel-Cox Log-Rank test (two-tailed); no comparisons were p<0.05.

FIG. 8 shows T cell response to anti-CD3/CD28 ligation and survival in patients treated with Provenge in accordance with one embodiment of the present disclosure. Survival comparison by race (Top left), or comparison by median fold mock induction of each cytokine shown after T cell (CD3 and CD28 ligation) stimulation. P value is from Mantel-Cox Log-Rank test (two-tailed); no comparisons were p<0.05.

FIG. 9 shows distinct innate inflammatory responses in PBMCs to in vitro challenge with PVSRIPO identify patients surviving longer after clinical PVSRIPO therapy in accordance with one embodiment of the present disclosure. PBMCs (5×10⁵) acquired about two weeks prior to PVSRIPO infusion were challenged with mock or PVSRIPO (MOI 10, 5×10⁶ plaque forming units) in vitro for 24 hours. Supernatant cytokine secretion was measured using Biolegend Human Antiviral Legendplex per manufacturer's instructions. Cytokines induced >2-fold mean for the cohort are included for analysis. Cytokine induction is shown for patients surviving >18 months (a) or >24 months (b) post-PVSRIPO administration; p values denote unpaired t-test (two, tailed) and brackets represent mean −/+SEM. (c) Kaplan-Meier plots are shown, stratifying survival by median induction for each cytokine as shown; p values denote Mantel-Cox log-rank test.

FIG. 10 shows data that patients surviving >18 months had a higher anti-polio neutralizing antibody titers in patient serum at baseline prior to therapy in accordance with one embodiment of the present disclosure. Polio neutralization titers determined prior to patient enrollment or boosting with polio vaccine were compared between patients living <18 months versus >18 months (OS) after PVSRIPO infusion. Panel A shows the results in the phase I trial and Panel B shows results in the Phase II trial. P values denote two-tailed unpaired t-test.

FIG. 11 shows data of an assessment of baseline peripheral immune cell function in response to the oncolytic poliovirus PVSRIPO in melanoma patients. PBMCs from the pre-treatment time point were challenged with laboratory grade PVSRIPO in vitro for 24 hours. Supernatant was tested for pro-inflammatory cytokines as indicated. Patients in red (left of each graph) have median RFS of 2.3 years after lerapolturev and patients in black (right of each graph) have median PFS 1.6 months after lerapolturev. Data brackets indicate mean −/+SEM; p values are from unpaired t-test.

FIG. 12 shows data of an assessment of peripheral immune cell function in response to the oncolytic poliovirus PVSRIPO in melanoma patients. PBMCs from the pre-treatment time point were challenged with the immune agonists indicated in the figure in vitro for 24 hours. Supernatant was tested for pro-inflammatory cytokines. Patients in red (right side of each pair) have median RFS of 2.3 years after lerapolturev and patients in black (left side of each pair) have median PFS 1.6 months after lerapolturev.

FIG. 13 shows peripheral immune cell function at baseline in mock-treated cells. PBMCs from the pre-treatment time point were mock-treated in vitro for 24 hours. Supernatant was tested for pro-inflammatory cytokines. FIG. 13 shows baseline values from mock treated samples. Patients in red have median RFS of 2.3 years after lerapolturev and patients in black have median PFS 1.6 months after lerapolturev.

### DETAILED DESCRIPTION

### I. Cytokine Production Indicative of Responsiveness to Immunotherapy

The present disclosure provides a method comprising contacting a patient sample with an innate immune agonist, and measuring the level of at least one cytokine produced by cells in the sample after the contacting step. The level of the cytokine produced is indicative of innate immune competence and responsiveness of the patient to immunotherapy. The level of cytokine may be compared to a reference level of cytokine produced by cells that was determined to indicate responsiveness of the cells and the cancer to immunotherapy. The method may be used to determine the immune proficiency of a patient, or a tissue therein, to respond to an innate immunity stimulus, immunotherapeutic, or immunotherapy. In the Examples the inventors show that in subjects with distinct cancers, responsiveness to an innate immune agonist in a peripheral blood sample from the patient can be measured via production of cytokines and correlates with that patient's cancer being responsive to immunotherapeutics. This provides a relatively non-invasive and cost effective means of determining which patients will benefit from an immunotherapeutic approach to treatment of their cancer. Accordingly, the present disclosure provides an in vitro immune competence test that allows for determining if a subject with cancer will be responsive to immunotherapies including oncolytic viral or checkpoint inhibitor immunotherapies. The findings provided here demonstrate that the responsiveness of the cancer to such immunotherapies can be evaluated via a relatively simple blood-based test for responsiveness to innate immune agonists. As shown in the Examples herein, such assessments of the cytokine production of a biological sample, such as peripheral blood or tumor biopsy, predicts responsiveness of the patient or tumor to a treatment, such as comprising an immunotherapy.

In the Examples, the inventors demonstrate that blood and tumor samples from patients with melanoma had similar immune responsiveness when contacted with a TLR4 agonist (lipopolysaccharide (LPS)) or a positive sense RNA virus and produced similar levels of IL-12 and TNF-α in response to these immune agonists.

In patients diagnosed with pancreatic cancer, the innate immune response to a positive stranded RNA virus in a sample of patient blood correlated to the immune responsiveness of the tumor demonstrating that the peripheral blood immune responsiveness correlates to the immune responsiveness of the tumor.

The inventors further demonstrate in the Examples that the correlation of immune responsiveness in the cancer and the peripheral blood of a patient is also indicative of the responsiveness of the cancer to an immunotherapeutic cancer treatment. The inventors show that those patients demonstrating immune responsiveness in peripheral blood samples (increased IFN-β after stimulation with a TLR1/2 agonist) were also more responsive to a prostate cancer therapy and survived longer. Similar results were observed in melanoma, where pretherapy peripheral blood mononuclear cells from patients with melanoma were contacted with the PVSRIPO oncolytic virus, and those patients whose cells were able to produce a strong IFN response in the in vitro assay associated with longer survival after PVSRIPO therapy.

As used herein, the term immune agonist refers to an agent or material that is capable of stimulating an immune signaling pathway, including in vitro. Typically, an immune agonist is a molecule or agent that interacts with and initiates signaling via an immune receptor, such as an adaptive or innate immune receptor. In some embodiments, the immune agonist is an innate immune agonist. An innate immune agonist is an agent that interacts with and initiates signaling via a receptor of the innate immune system. Non-limiting examples of receptors of the innate immune system include pattern recognition receptors (PRRs), toll-like receptors (TLRs), C-type lectin receptors (CLR), and cytosolic nucleic acid sensors. A pattern recognition receptor agonist refers to a type of innate immune agonist which binds and stimulates a pattern recognition receptor. For example, a STING/TMEM173 agonist refers to a type of innate immune agonist which binds and stimulates signaling via the STING/TMEM173 receptor, a type of PRR. Examples include, but are not limited to, oncolytic viruses, STING agonists, TLR agonists and combinations thereof. Examples of innate immune agonists are known to those of skill in the art and include but are not limited to synthetic double stranded RNAs (e.g. Poly I:C, Poly A:U) or DNAs, flagellin, zymosan, LPS, PAM3CSK4, viruses, bacteria, and Imiquimod/R848. In certain embodiments, the innate immune agonist comprises lipopolysaccharide (LPS). In other embodiments, the innage immune agonist comprises +-stranded RNA virus. A T cell agonist refers to a type of agonist which binds and stimulates a receptor on the surface of a T cell.

“Contacting” as used herein, e.g., as in “contacting a sample” refers to contacting a sample directly or indirectly in vitro or ex vivo. Contacting a sample may include addition of one or more compounds to a sample. Contacting encompasses administration to a solution, cell, tissue, mammal, subject, patient, or human. Further, contacting a cell includes adding an agent to a cell culture.

As used herein, the term “reference level” with regard to a cytokine level in a sample refers to a positive signal which may also mean a level above which a disease is responsive to immunotherapy. The reference level can be determined for each cytokine empirically and may depend on the innate immune agonist used to initiate the cytokine induction. In some embodiments, the disease is a cancer and the immunotherapy is an anti-cancer immunotherapy.

Cytokines are a broad group of small proteins that function in cell signaling by binding a cell surface receptor of the immune system. Typically, cytokines are peptides or polypeptides of around 5 to 30 kDa that often play important roles in the immune system, e.g., during immune responses to inflammation, infection, trauma, sepsis, and cancer. Non-limiting examples of cytokines include chemokines, interferons, interleukins, lymphokines, and tumor necrosis factors.

In some embodiments, the cytokine is a pro-inflammatory cytokine. In some embodiments, the cytokine is selected from: TNFα, IL-12, IFN-α, IFN-β, IFN-γ, IFN-λ2, IL-28, IL-29, CXCL10, GMCSF, IL-1β, IL-6, IFN-γ1, IL-8, and IL-10.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a human. In other embodiments, the subject comprises a human subject suffering from, or believed to be suffering from, a cancer.

As is known in the art, a cancer is generally considered as uncontrolled cell growth. In some embodiments, the cancer comprises cancer in the form of a tumor. The methods of the present disclosure can be used for assessing and determining treatment options for any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

A patient sample or sample obtained from a patient refers to a biological sample. The term “biological sample” or “sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample comprises a biopsy (such as a tumor biopsy). In other embodiments, the biological sample comprises a blood sample or the peripheral blood mononuclear cells (PBMCs) isolated from a blood sample. A biological sample may be obtained directly from a subject (e.g., by blood or tissue sampling) or from a third party (e.g., received from an intermediary, such as a healthcare provider or lab technician). In some embodiments, the biological sample comprises a blood (e.g., peripheral blood) sample, serum sample, and/or a tumor biopsy.

As used herein, the phrase “responsiveness of a patient to a therapy” refers to the patient (or disease of the patient) positively responding the therapy, such as, e.g., as evidenced by the alleviation or prevention of a symptom(s), slowing or stopping a progression or worsening of a disease, disorder, or condition and/or the remission of the disease, disorder or condition (e.g., cancer). For example, the extension of life span or an improvement in quality of life may represent a positive response.

As used herein, “treatment,” “therapy” and/or “therapy regimen” refer to the clinical intervention made in response to a disease, disorder or physiological condition (e.g., cancer) manifested by a patient or to which a patient may be susceptible. The aim of treatment includes the alleviation or prevention of symptoms, slowing or stopping the progression or worsening of a disease, disorder, or condition and/or the remission of the disease, disorder or condition (e.g., cancer). An “anticancer therapy” refers to any commonly administered therapies to treat a cancer in a subject. An example is immunotherapy. The appropriate therapy is dependent on numerous factors, such as age of the patient, type of cancer, location of the tumor, stage of the cancer etc. and can be readily determined by one skilled in the art.

As used herein, the terms “prevent,” “preventing,” “prevention,” “prophylactic treatment” and the like refer to reducing the probability of developing a disease, disorder or condition (e.g., cancer) in a subject, who does not have, but is at risk of or susceptible to developing a disease, disorder or condition.

The term “immunotherapy” refers to a therapy which stimulates the recipient's immune system in some way to provide a benefit to the subject. An anticancer immunotherapy is an immunotherapy which stimulates the recipient's immune system to treat a cancer. An immunotherapy can be active or passive and can operate via the innate and/or adaptive immune systems. An example of an active immunotherapy is administration of a cancer vaccine or CAR-T cell, both of which can target a specific cancer antigen. An example of a passive immunotherapy is an immune checkpoint inhibitor, which relieves repression of certain aspects of the immune system but does not necessarily target a specific antigen or cancer cell type. Another example of a passive immunotherapy is administration of a cytokine which stimulates the immune system and/or specific immune responses. Administration of an oncolytic virus is an additional type of immunotherapy which may act as both passive and active immunotherapy. Generally, the administration of a foreign or non-self antigen has the potential to stimulate the immune system.

As used herein, the term “administering” an agent, such as a therapeutic entity to a subject or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. The term “effective amount” or “therapeutically effective amount” refers to an amount sufficient to effect beneficial or desirable biological and/or clinical results.

A cytokine may be detected or cytokine level may be measured using any cytokine assay known in the art and/or described herein. Non-limiting examples of a cytokine assay include enzyme-linked absorbent immunoassays (ELISAs) (including, e.g., an enzyme-linked immunosorbent spot (ELISpot) assay), fluorescent immunoassays such as intracellular cytokine staining, antibody array technologies, radioimmunoassays, surface plasmon resonance-based detection methods, and other immunoassays, such as using a cytokine capture antibody agent immobilized on a microbead (see e.g., Siebert J, Walker E, Immunotherapy 2: 799-816 (2010); Greenplate A, et al., Eur J Cancer 61: 77-84 (2016); Diefenbach C et al., Blood 134: 3980 (2019); Ji, A et al., Cell 182: 497-514 (2020)). One or more cytokines of interest may be detected using a multiplex immunoassay (see e.g., Young, H et al., Methods Mol Biol 511: 85-105 (2009); Chowdhury F et al., J Immunol Methods 340, 55-64 (2009)). A cytokine of interest may be detected using an anti-cytokine antibody array. A cytokine may be detected using a cytokine capture bead array, which may be processed using a flow cytometry method. Another example is using Luminex instrumentation to process a bead array cytokine detection assay. A cytokine may be detected or cytokine level may be measured using a cytokine functional assay. Such methods are available to those skilled in the art.

A cytokine may be detected or cytokine level may be measured in a cell, collection of cells, or tissue using an intracellular cytokine staining (ICS) assay (see e.g. Foote J et al., Methods in Enzymology 631, 1-20 (2020)). For example, cells are treated with a transport inhibitor (e.g. brefeldin A) to retain any cytokines in the cells. Then the cells are labeled with one or more probes (e.g an anti-cytokine antibody conjugated to a detectable label) each specific to a cytokine. Flow cytometry or fluorescence activated cell scanning (FACS) techniques may be used to provide quantitative information via an ICS technique.

Cytokine levels may be measured using a method of detecting a mRNA encoding the cytokine, such as, e.g. using a RT-PCR assay. (See e.g. Mocellin S et al., J Immunol Methods 280: 1-11 (2003)). Non-limiting techniques for more quantitative cytokine detections include quantitative polymerase chain reaction (qPCR) and next generation sequencing (NGS) of RNA (RNA-seq). (See e.g., Kukurba K et al., Cold Spring Harb Protoc 2015: 951-69 (2015); Choi J et al., Cells 9: 1130 (2020)). Suitable next generation sequencing technologies are also widely available and considered within the scope of the present disclosure. Non-limiting examples of NGS include the 454 Life Sciences platform (Roche, Branford, CT); Illumina's Genome Analyzer, GoldenGate Methylation Assay, or Infinium Methylation Assays, e.g., Infinium HumanMethylation 27K BeadArray or VeraCode GoldenGate methylation array (Illumina, San Diego, CA); QX200™ Droplet Digital™ PCR System from Bio-Rad; or DNA Sequencing by Ligation, SOLID System (Applied Biosystems/Life Technologies, Waltham, MA); the Helicos True Single Molecule DNA sequencing technology (see e.g. Harris et al, Science 320, 106-109 (2008)), the single molecule, real-time (SMRT™) technology of Pacific Biosciences (Menlo Park, CA), and solid state nanopore sequencing (Soni and Meller, Clin Chem. 53, 1996-2001 (2007)); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys).

Cytokine levels may be determined at the single cell level, such as, e.g. using an ELISpot or ICS assay. (See e.g., Casanovas R et al., J Clin Oncol 25, 1732-40 (2007); Tan A, et al., B. J. Hepatol 52, 330-9 (2010); Siebert J. Walker E, Immunotherapy 2: 799-816 (2010)). For example, IsoLight, IsoCode chip, or Berkeley Light Switch methods can be used to detect cytokines at the single cell level (see e.g., Thurin M et al., Biomarkers for Immunotherapy of Caner: Methods and Protocols; 2020).

In some embodiments, the patient or subject has cancer. In some embodiments, the patient sample comprises a cancer cell. In some embodiments, the cancer is selected from: a brain cancer including but not limited to glioblastoma, astrocytoma, meningioma, medulloblastoma, craniopharyngioma, germinoma, pineoblastoma; breast cancer, including but not limited to estrogen-positive breast cancer, HER-positive breast cancer, HER-negative breast cancer, and triple negative breast cancer; bladder cancer including but not limited to muscle invasive bladder cancer and non-muscle invasive bladder cancer; lung cancer including but not limited to non-small cell lung cancer and small cell lung cancer; head and neck cancer; esophageal cancer; colorectal cancer; gastric cancer; liver cancer; kidney cancer; skin cancer, including basal cell carcinoma, Merkel cell carcinoma, melanoma, and squamous cell carcinoma; endometrial cancer; cervical cancer; or ovarian cancer.

The methods provided herein may be used to select patients for immunotherapeutic treatment of their cancer. Patients showing responsiveness to an innate immune agonists may be admininstereds an immunotherapy. In some embodiments, the immunotherapy comprises an agent selected from an immune checkpoint inhibitor, vaccine, adjuvant, cytokine, human cell therapy, microorganism, and virus. In some embodiments, the vaccine is a cancer vaccine such as, e.g., a Provenge, a cell-based cancer vaccine. In some embodiments, the human cell therapy comprises a T cell, a CAR-T cell, an engineered NK cell, an engineered Treg cell, or any type of immune cell. In some embodiments, the microorganism comprises an engineered strain of bacterium. In some embodiments, the immunotherapy comprises a PD-1 inhibitor, PD-L1 inhibitor, PD-L2 inhibitor, CTLA-4 inhibitor, and/or LAG-3 inhibitor.

In some embodiments, the method further comprises administering an immunotherapy to the patient and treating the patient with at least one additional therapeutic modality in addition to the immunotherapy. In some embodiments, the additional therapeutic modality comprises administering an oncolytic virus to the patient. In some embodiments, the immunotherapy comprises administering an oncolytic virus to patient diagnosed with cancer. In some embodiments, the oncolytic virus is selected from a poliovirus, adenovirus, HSV-1 virus, reovirus, poxvirus, Newcastle Disease virus, measles virus, Seneca Valley virus, hemagglutinating virus of Japan Envelope (HVJ-E) virus, herpes virus, parvovirus, retrovirus, PVS-RIPO, paleorep, GEN0101, seprehvir talimogene laherparepvec, adenovirus VCN-01, adenovirus ICORVIR-5, HF10, GL-ONC1, DNX-2401, and enadenotucirev, or a derivative of any of the aforementioned. Virus derivatives include but is not limited to inactive, recombinant, and genetically engineered viruses. In some embodiments, the oncolytic virus is a polio virus and/or a polio virus derivative. In some embodiments, the polio virus derivative is PVS-RIPO (Lerapolturev).

Any innate immune agonist may be used to achieve the methods provided herein. Examples include, but are not limited to, oncolytic viruses, STING agonists, TLR agonists and combinations thereof. In some embodiments, the innate immune agonist is selected from the group consisting of a pattern recognition receptor (PRR) agonist, a microorganism or antigen thereof, a virus or antigen thereof, a STING/TMEM173 agonist and a T cell agonist. In some embodiments, the innate immune agonist is +-stranded RNA virus or an antigenic derivative thereof. In some embodiments, the innate immune agonist is an oncolytic virus or an antigenic derivative thereof. In some embodiments, the pattern recognition receptor agonist is selected from a toll-like receptor agonist, C-type lectin receptor agonist, NOD-like receptor agonist, and RIG-I-like receptor (RLR) agonist. In some embodiments, the toll-like receptor agonist is selected from a TLR4 agonist, TLR1/2 agonist, and a TLR7/8 agonist. In some embodiments, the innate immune agonist comprises lipopolysaccharide (LPS).

In other embodiments provided herein, the sample may be contacted with more than one innate immune agonist, such as two, three or even four or more agonists. The measuring step may comprise measuring the level of at least two, three, four or more inflammatory cytokines.

In some embodiments, the patient has melanoma, the innate immune agonist is LPS or +-RNA virus, and the proinflammatory cytokine measured is TNFα.

In some embodiments, the patient has pancreatic cancer, the innate immune agonist is a viral antigen or RNA virus, and the proinflammatory cytokine is TNFα or CXCL10.

In some embodiments, the patient has pancreatic cancer, the innate immune agonist is a TLR1/2 agonist (such as PAM3CSK4), and the proinflammatory cytokine is IFN-β.

In some embodiments, the present disclosure provides an in vitro immune competence blood test that allows for determining if a cancer is likely to be responsive to immunotherapeutics. As shown in the Examples herein, such assessments of the immune function of a biological sample, such as peripheral blood or tumor biopsy, predicts responsiveness of the tumor to cancer treatment, such as comprising an immunotherapy.

In another aspect, the present disclosure provides a method for determining the immune status of a tumor in a subject, the method comprising, consisting of, or consisting essentially of (i) obtaining a biological sample from the subject; (ii) introducing to the sample an innate immune agonist; (iii) measuring the levels of TNF-α and IL-12 produced by the sample and comparing to a control; and (iv) implementing an appropriate anti-cancer treatment protocol based on the results.

Another aspect of the present disclosure provides a method for predicting the immune status of a tumor in a subject, the method comprising, consisting of, or consisting essentially of (i) obtaining a biological sample from the subject; (ii) introducing to the sample an innate immune agonist; (iii) measuring the levels of TNF-α and IL-12 produced by the sample and comparing to a control; and (iv) implementing an appropriate anti-cancer treatment protocol based on the results.

One aspect of the present disclosure provides a method for determining the immune status of a tumor in a subject, the method comprising, consisting of, or consisting essentially of (i) obtaining a biological sample from the subject; (ii) introducing to the sample an innate immune agonist; (iii) measuring the levels of TNF-α and IL-12 produced by the sample and comparing to a control; and (iv) implementing an appropriate anti-cancer treatment protocol based on the results.

Another aspect of the present disclosure provides a method for predicting the immune status a tumor in a subject, the method comprising, consisting of, or consisting essentially of (i) obtaining a biological sample from the subject: (ii) introducing to the sample an innate immune agonist; (iii) measuring the levels of TNF-α and IL-12 produced by the sample and comparing to a control; and (iv) implementing an appropriate anti-cancer treatment protocol based on the results.

Other aspects of the present disclosure provide kits for determining the immune activity of a tumor in a subject comprising, consisting of, or consisting essentially of a means of collecting a biological sample, required reagents, and/or instructions for use. The kit may comprise a means of collecting a biological sample, such as a syringe and collection bottle, required reagents, such as buffers, anticoagulants and the like, and instructions for use.

### II. Anti-Virus Antibody Level Indicative of Responsiveness to a Virotherapy

In another aspect, the present disclosure provides a method of treating a subject with an oncolytic viral therapy, the method comprising: (a) obtaining a serum sample from a subject diagnosed with a cancer; (b) detecting the level of antibody specific to a viral antigen in the serum sample; (c) comparing the level of the antibody in the sample to a reference level; and (d) treating the subject with an oncolytic viral therapy when the level of the antibody in the sample is above the reference level.

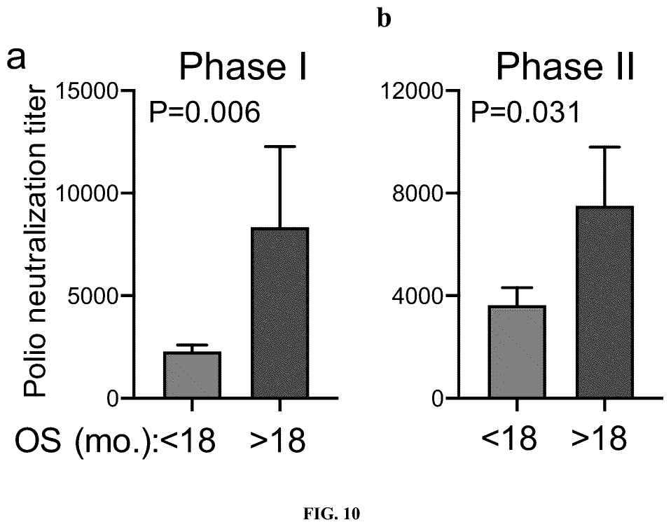

It was surprisingly discovered that subjects having high pre-existing neutralizing antibody titers to an oncolytic virus, or related virus thereto, prior to administration of the oncolytic virus or a related booster virus for the treatment of a tumor have significantly improved therapeutic outcomes, including overall survival (OS) improvement. For example, it has been discovered that subjects with recurrent glioblastoma multiforma (rGBM) having high anti-polio neutralizing antibody titers at baseline (e.g., >1:4,000) prior to administration of polio virotherapy (e.g., PVS-RIPO) experienced significantly longer overall survival than subjects having lower anti-polio neutralizing antibody titers at baseline (e.g., >18-months for higher titers vs. <18-months for lower titers) (see FIG. 10 and Example 3). While it would be anticipated that pre-existing immunity to PVSRIPO impedes antitumor therapy, the evidence described above and provided in the examples indicates that immunological “recall”, or reactivation of memory T cells, may mediate anti-tumor effects. In some aspects, this surprising discovery allows for the identification of subjects most likely to benefit from the administration of a virotherapy.

An oncolytic viral therapy is a treatment using an oncolytic virus, typically to treat a cancer, tumor, or other uncontrolled growth. The term “oncolytic virus” refers to a virus that preferentially infects and kills cancer or tumor cells by lysis or programmed cell death. An oncolytic virus may be synthetic and/or genetically engineered. An oncolytic virus may have a wild-type genome. An example of an oncolytic virus used in virotherapies for cancer is talimogene laherparepvec (T-VEC, Imlygic™, OncoVex™), which is based on a herpes simplex virus. The oncolytic virus can be, for example but not limited to, an oncolytic poliovirus (PVS-RIPO), an oncolytic adenovirus, oncolytic HSV-1, an oncolytic reovirus, an oncolytic poxvirus, an oncolytic Newcastle Disease virus, an oncolytic measles virus, an onvolytic Seneca Valley virus, a humagluttinating virus of Japan Envelope (HVJ-E) virus, an oncolytic gamma-herpes virus, an oncolytic parvovirus, or an oncolytic retrovirus. Oncolytic viruses include, but are not limited to, PVS-RIPO, paleorep (Reolysin, Oncolytics Biotech), hemagglutinating virus of Japan-envelope (GEN0101), seprehvir, talimogene laherparepvec (T-VEC), adenovirus VCN-01, adenovirus ICORVIR-5, HF10, GL-ONC1, DNX-2401, and enadenotucirev.

As used herein, the term “reference level” with regard to a viral neutralizing antibody level in sample may refer to a level that is increased compared to patients which are non-responsive to an oncolytic viral therapy. As used herein, the term “reference level” with regard to a viral neutralizing antibody level in a serum sample may also refer to a level that allows for at least 50% plaque neutralization at a 1:5,000 dilution of serum.

Some methods provided herein comprise detecting the level of antibody specific to a viral antigen in a serum sample from a patient. An antibody level in a sample can be detected according to any suitable method known in the art (see e.g., Boone et al., Conventional and Enhanced Plaque Neutralization Assay for Polio Antibody. J. Virol. Methods, Volume 6, Issue 4, April 1983, Pages 193-202).

An antibody titer is functionally defined by the working concentration or dilution of an antibody sample that is necessary to achieve a minimum level of specific detection in a given assay. The skilled worker knows how to establish an exact minimum acceptable value using a method known in the art, such as, e.g., by reference to a statistically significant signal-to-noise ratio. Non-limiting examples of methods for measuring an antibody titer include ELISA, ELISpot assays, fluorescent immunoassays, antibody array technologies, radioimmunoassays, and other immunoassays known in the art.

In some embodiments, the antibody titer is determined in a standard antibody neutralization assay. In some embodiments, the antibody titer level against the virus indicative of a subject being responsive to or benefiting from a virotherapy is at least about 1:4000, at least about 1:4500, at least about 1:5000, at least about 1:5500, at least about 1:6000, at least about 1:6500, at least about 1:7000, or greater as assessed, e.g., in a plaque neutralization assay.

In one aspect, provided herein is a method of treating a subject with cancer comprising:
i) determining an antibody titer to a virus comprising the virotherapy, or a virus related thereto, wherein the antibody titer is determined from a biological sample of the subject, and wherein the antibody titer is determined at baseline before the administration of the virotherapy or a booster virus related thereto;
ii) comparing the antibody titer to the virus in the biological sample of the subject to a reference value, wherein the reference value indicates whether the antibody titer is indicative of the subject being response to the virotherapy; and,
iii) if the antibody titer of the subject compared to the reference level indicates that the subject being responsive to the virotherapy, administering to the subject the virotherapy.

In one aspect, provided herein is a method of determining whether a subject with cancer will benefit from or be responsive to a virotherapy comprising:
i) determining an antibody titer to a virus comprising the virotherapy, or a virus related thereto, wherein the antibody titer is determined from a biological sample of the subject, and wherein the antibody titer is determined at baseline before the administration of the virotherapy or a booster virus related thereto;
ii) comparing the antibody titer in the biological sample of the subject to a reference value, wherein the reference value indicates whether the antibody titer is indicative of the subject being response to the virotherapy; and,
iii) if the antibody titer of the subject compared to the reference level indicates that the subject being responsive to the virotherapy, then the subject would be responsive to or benefit from the virotherapy.

In one aspect, provided herein is a method of selecting a subject with cancer for virotherapy comprising:
i) determining an antibody titer to a virus comprising the virotherapy, or a virus related thereto, wherein the antibody titer is determined from a biological sample of the subject, and wherein the antibody titer is determined at baseline before the administration of the virotherapy or a booster virus related thereto;
ii) comparing the antibody titer to the virus in the biological sample of the subject to a reference value, wherein the reference value indicates whether the antibody titer level is indicative of the subject being response to the virotherapy; and,
iii) if the antibody titer of the subject compared to the reference level indicates that the subject being responsive to the virotherapy, administering to the subject the virotherapy.

In one aspect, provided herein is a method of determining the efficacy of a virotherapy in a subject with cancer comprising:
i) determining an antibody titer to a virus comprising the virotherapy, or a virus related thereto, wherein the antibody titer is determined from a biological sample of the subject, and wherein the antibody titer is determined at baseline before the administration of the virotherapy or a booster virus related thereto;
ii) comparing the antibody titer to the virus in the biological sample of the subject to a reference value, wherein the reference value indicates whether the antibody titer level is indicative of the subject being response to the virotherapy; and,
iii) if the antibody titer of the subject compared to the reference level indicates that the subject being responsive to the virotherapy, administering to the subject the virotherapy.

The virotherapy of the above-described aspects can be, for example, an oncolytic virus. In particular embodiments, the oncolytic virus is a oncolytic poliovirus or poliovirus-derived oncolytic virus. In a particular embodiment, the oncolytic virus is the oncolytic poliovirus-derived PVS-RIPO. The cancer of the above-described aspects can be any suitable cancer susceptible to virotherapy. In some embodiments, the cancer is a solid tumor. In some embodiments, the solid tumor is amenable to intratumoral injection. In some embodiments, the cancer expresses the CD155 (poliovirus receptor). In some embodiments, the cancer is not an Epstein Barr-virus (EBV)-related malignancy. In some embodiments, the cancer is selected from: a brain cancer including but not limited to glioblastoma, astrocytoma, meningioma, medulloblastoma, craniopharyngioma, germinoma, pineoblastoma; breast cancer, including but not limited to estrogen-positive breast cancer, HER-positive breast cancer, HER-negative breast cancer, and triple negative breast cancer; bladder cancer including but not limited to muscle invasive bladder cancer and non-muscle invasive bladder cancer; lung cancer including but not limited to non-small cell lung cancer and small cell lung cancer; head and neck cancer; esophageal cancer; colorectal cancer; gastric cancer; liver cancer; pancreatic cancer; prostrate cancer; kidney cancer; skin cancer, including basal cell carcinoma, Merkel cell carcinoma, melanoma, and squamous cell carcinoma; endometrial cancer; cervical cancer; or ovarian cancer.

In some embodiments of the above aspects, the benefit from or response to the virotherapy in the subject is an improvement or increase in overall survival (OS), progression free survival (PFS), objective response rate (ORR), Duration of Objective Response (DOR), and/or Clinical Benefit Rate (CBR) compared to a subject that 1) does not receive virotherapy and/or 2) a subject whose antibody titer level is not indicative of such benefit or response.

In some embodiments of the above aspects, the antibody titer is determined for the virus comprising the virotherapy. In an alternative embodiment, the antibody titer is determined for a virus from which the virotherapy is derived, for example a parental virus from which the virotherapy virus is derived from or related to. The antibody titer can be measured according to known methods in the art, (see e.g., Boone et al., Conventional and Enhanced Plaque Neutralization Assay for Polio Antibody. J. Virol. Methods, Volume 6, Issue 4, April 1983, Pages 193-202). In some embodiments, the antibody titer is determined in a standard antibody neutralization assay. In some embodiments, the antibody titer level against the virus indicative of a subject being responsive to or benefiting from a virotherapy is at least about 1:4000, at least about 1:4500, at least about 1:5000, at least about 1:5500, at least about 1:6000, at least about 1:6500, at least about 1:7000, or greater as assessed, e.g., in a plaque neutralization assay. In particular embodiments, the virotherapy is the oncolytic polio-virus derived PVS-RIPO and the antibody titer is determined for poliovirus or PVS-RIPO.

The virotherapy of the above-described aspects can be, for example, an oncolytic virus. The oncolytic virus can be, for example but not limited to, an oncolytic poliovirus, an oncolytic adenovirus, oncolytic HSV-1, an oncolytic reovirus, an oncolytic poxvirus, an oncolytic Newcastle Disease virus, an oncolytic measles virus, an onvolytic Seneca Valley virus, a humagluttinating virus of Japan Envelope (HVJ-E) virus, an oncolytic gamma-herpes virus, an oncolytic parvovirus, or an oncolytic retrovirus. Oncolytic viruses include, but are not limited to, PVS-RIPO, paleorep (Reolysin, Oncolytics Biotech), hemagglutinating virus of Japan-envelope (GEN0101), seprehvir, talimogene laherparepvec (T-VEC), adenovirus VCN-01, adenovirus ICORVIR-5, HF10, GL-ONC1, DNX-2401, and enadenotucirev.

In particular embodiments, the oncolytic virus is an oncolytic poliovirus or poliovirus-derived oncolytic virus. In a particular embodiment, the oncolytic virus is the oncolytic poliovirus-derived PVS-RIPO. PVSRIPO is being tested in multi-institutional clinical trials for recurrent glioblastoma (NCT04479241), unresectable, PD-1 refractory melanoma (NCT04577807), and in solid tumors (NCT0469069), including in combination with anti-PD-1/L1 checkpoint inhibitors.

The cancer of the above-described aspects can be any suitable cancer susceptible to virotherapy. In some embodiments, the cancer is a solid tumor. In some embodiments, the solid tumor is amenable to intratumoral injection. In some embodiments, the cancer is not an Epstein Barr-virus (EBV)-related malignancy, e.g., nasopharyngeal carcinoma (NPC), certain B cell lymphomas, certain tumors derived from T-cells and NK cells (see, e.g., Delecluse et al., Epstein-Barr virus-associated tumours: an update for the attention of the working pathologist. J Clin Pathol. 2007 December; 60(12): 1358-1364).

In some embodiments of the above aspects, the virotherapy is PVS-RIPO, and the cancer is glioblastoma multiforma. In some embodiments, the virotherapy is PVS-RIPO, and the cancer is melanoma. In some embodiments, the virotherapy is PVS-RIPO, and the cancer is head and neck cancer (H&NC). In some embodiments, the virotherapy is PVS-RIPO, and the cancer is endometrial cancer. In some embodiments, the virotherapy is PVS-RIPO, and the cancer is esophageal cancer. In some embodiments, the virotherapy is PVS-RIPO, and the cancer is bladder cancer. In some embodiments, the virotherapy is PVS-RIPO, and the cancer is non-muscle invasive bladder cancer (NMIBC). In some embodiments, the virotherapy is PVS-RIPO, and the cancer is muscle invasive bladder cancer (MIBC). In some embodiments, the virotherapy is PVS-RIPO, and the cancer is ovarian cancer. In some embodiments, the virotherapy is PVS-RIPO, and the cancer is breast cancer. In some embodiments, the virotherapy is PVS-RIPO, and the cancer is cervical cancer. In some embodiments, the measured antibody titer is to poliovirus.

In some embodiments of the above aspects, the benefit from or response to the virotherapy in the subject is an improvement or increase in overall survival (OS), progression free survival (PFS), objective response rate (ORR), Duration of Objective Response (DOR), and/or Clinical Benefit Rate (CBR) compared to a subject that 1) does not receive virotherapy and/or 2) a subject whose antibody titer level is not indicative of such benefit or response. Overall Survival (OS) is generally calculated as the time (months) from the date of the onset of protocol administration to the date of death due to any cause. Progression free survival (PFS) is generally defined as the time (number of months) from date of protocol administration until the date of documented radiologic disease progression or death from any cause. Objective response rate (ORR) is generally defined as the proportion of patients with tumor size reduction of a predefined amount and for a minimum time period. Objective response (OR) includes a complete response (CR), which is the disappearance of all signs of the tumor in response to treatment and a partial response (PR), which is a decrease in the size of a tumor in response to treatment. In some embodiments, the objective response (OR) is a complete response (CR). In some embodiments, the objective response (OR) is a partial response (PR). The ORR is an important parameter to demonstrate the efficacy of a treatment and it serves as a primary or secondary end-point in many clinical trials. Duration of Objective Response (DOR) is generally defined as the time between first objective response of CR or PR and the first date that progressive disease is objectively documented or death, whichever comes first. Clinical Benefit Rate (CBR) is generally defined as the proportion of patients with CR (any duration), PR (any duration) or stable disease (SD) (≥6 months). Methods of accessing increased OS and/or PFS and/or ORR and/or DOR and/or CBR are well known in the art and include, for example RECIST v1.1 (Eisenhauer et al. New response evaluation criteria in solid tumors: revised RECIST guideline (version 1.1). Eur J Cancer. 2009; 45: 228-247) and World Health Organization (WHO) (World Health Organization. WHO Handbook for Reporting Results of Cancer Treatment. World Health Organization Offset Publication No. 48; Geneva (Switzerland), 1979). Statistical methods of measuring OR, PFS, ORR, DOR, and/or CBR are well known in the art and include, for example, the Clopper-Pearson Method (Clopper, C.; Pearson, E. S. (1934). “The use of confidence or fiducial limits illustrated in the case of the binomial”. Biometrika. 26 (4): 404-413. doi: 10.1093/biomet/26.4.404).

Standardized test for assessing an antibody titer in a biological sample, for example serum, blood, or other bodily fluid, are known in the art. One method for assessing neutralizing antibody titers to a virus utilizes a cell-based assay (see e.g., Boone et al., Conventional and Enhanced Plaque Neutralization Assay for Polio Antibody. J. of Virol. Methods. Volume 6, Issue 4, April 1983, Pages 193-202). Commercial cell-based assays assessing neutralizing antibody titers are available (see, e.g., Quest Diagnostics, Poliovirus (Types 1, 3) Antibodies, Neutralization, Test Code 94106). The cell-based assay quantitatively measures neutralizing antibody titers in patient sera by adding patient serum and a solution containing the virus to viral-susceptible cells, and analyzing the cells to determine if the vims can no longer infect the cells. In this cell-based assay, a reduction in viral induced cytotoxicity is a measure of neutralization activity in the sera. The strength of neutralization is reported in two ways: (i) the IC50 (e.g., half the cells are killed) or (ii) highest dilution at which neutralization activity disappears. One method for performing a plaque neutralization assay is further described in Terletskaia-Ladwig, E. et al., J Virol Methods 178: 124-8 (2011). In some embodiments, the virotherapy is the oncolytic polio-virus derived PVS-RIPO and the antibody titer is determined for poliovirus or PVS-RIPO, wherein an indication of response or benefit is present when the antibody titer is greater than at least 1:4000 as measured by a plaque neutralization assay.

The term “effective amount” or “therapeutically effective amount” refers to an amount sufficient to effect beneficial or desirable biological and/or clinical results.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like. As is known in the art, a cancer is generally considered as uncontrolled cell growth. In some embodiments, the cancer comprises cancer in the form of a tumor. The methods of the present disclosure can be used to treat any cancer, and any metastases thereof, including, but not limited to, carcinoma, lymphoma, blastoma, sarcoma, and leukemia More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

As used herein, the term “correlates” as between a specific cytokine level or a anti-viral antibody level and/or a therapeutic outcome of a subject providing a sample refers to an identifiable connection between an indicator in the sample of a subject and the subject's likelihood to respond to a class or type of therapy (e.g., immuotherapry or oncolytic virotherapy).

Unless otherwise defined, all technical terms used herein have the same meaning as commonly understood by one of ordinary skill in the art to which this disclosure belongs.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

An aspect of the present invention is provide by all that is described and illustrated herein, including any and all methods, processes, devices, systems, devices, kits, products, materials, compositions and/or uses shown and/or described expressly or by implication in the information provided herewith, including but not limited to features in the present disclosure that may be apparent and/or understood by those of skill in the art.

The following Examples are provided by way of illustration and not by way of limitation.

### Examples

### 1. Innate Immune Dysfunction in Blood as a Predictor of Tumor Immune Status

A. Study Using Blood and Tumor from Patients with Melanoma:

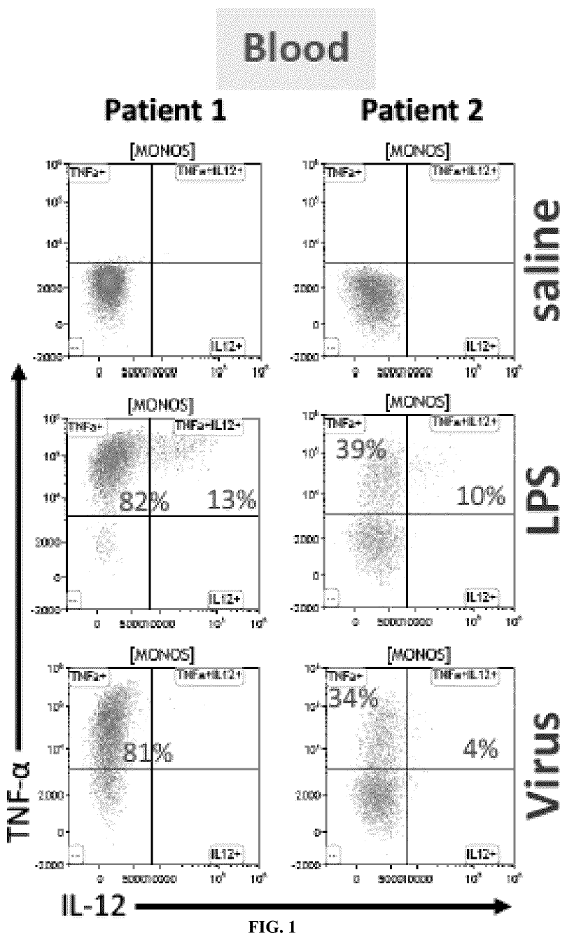

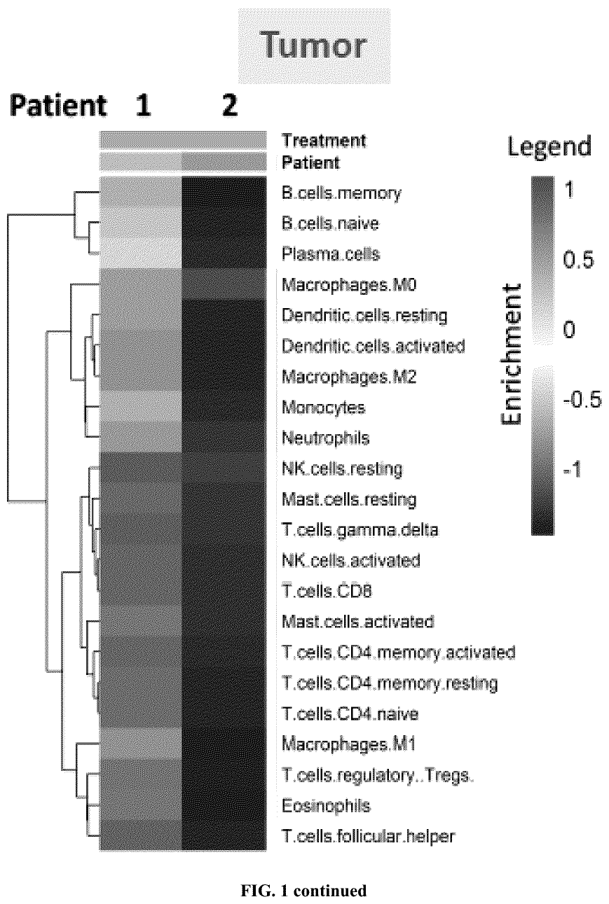

The relationship between peripheral innate immune function and the immune landscape of the tumor was examined. To study innate immune function, monocytes in blood were tested for their responsiveness to two innate immune adjuvants: (1) TLR4 agonist LPS and (2) +-stranded RNA virus. FIG. 1 demonstrates robust innate immune responsiveness to both innate immune adjuvants in patient 1 monocytes compared to attenuated responses to the adjuvants in patient 2. Remarkably, innate immune responsiveness in blood correlated with the immune landscape of tumor: patient 1 had an active tumor immune signature while patient 2 had no tumor immune activity.

B. Study Using Blood and Tumor from Patients with Pancreatic Cancer:

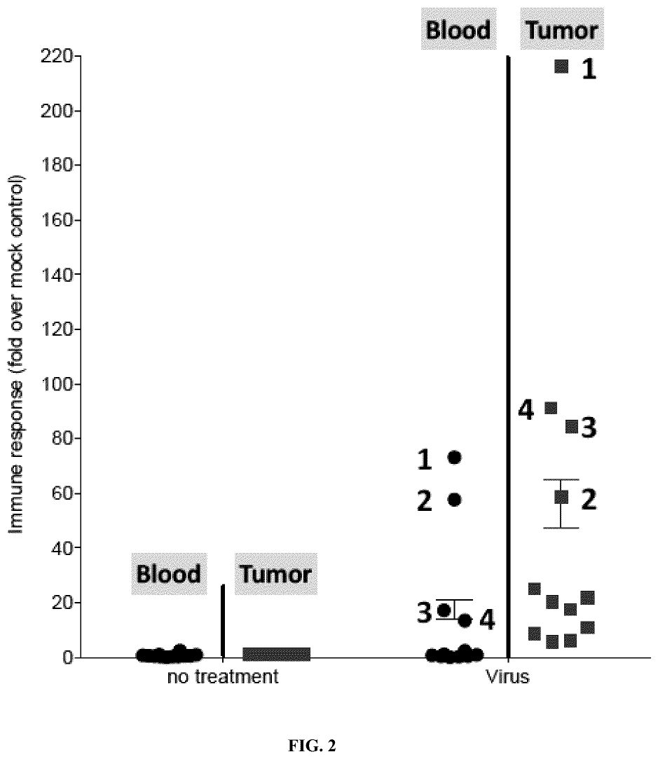

The hypothesis that innate immune response in blood will predict the immune response to an innate immune agonist in tumor was tested. This study was conducted using tumor and blood from patients with pancreatic cancer. Innate immune response to a +-stranded RNA virus in blood was compared to innate immune response to virus in the tumor. Untreated samples served as a baseline control. Data is presented in FIG. 2. Data shows immune response (TNFα production) in monocytes in blood and immune response in tumor and is shown as cytokine production (pg/ml) over mock control. Tumor immune function in response to innate immune agonist is determined using a tumor tissue slice assay. Tumor tissue is sliced and randomly assigned to 5×35 mm dishes and treated with mock (saline) or virus for 48 hours. Supernatant is analyzed for cytokine production (CXCL10) in response to virus. Peripheral (blood) monocytes from patients who had a positive response to the innate immune agonist (depicted as 1 through 4) was observed to have demonstrated a robust response to virus in the tumor.

2. Induction of IFN-β after TLR1/2 Stimulation Prior to Provenge Therapy Associates with Overall Survival in Prostate Cancer Patients

### A. Introduction

Provenge (Sipuleucel-T) is an autologous dendritic cell PAP antigen vaccine FDA approved for the treatment of PCa. Black or African American (AA) patients survive longer after Provenge therapy relative to Caucasian American (CA) PCa patients. PCa tumors from AA patients have also been shown to harbor higher chemokine and interferon signatures relative to tumors from CA patients. One possible explanation for these differences may be due to inherent, genetic differences in the immune system. Indeed, recent evidence indicates several genetic differences in African ancestral individuals vs Caucasians, particularly in the recognition of bacteria, mediated through innate immune pattern recognition receptor signaling. Indeed, stronger interferon responses after TLR1/2 activation via PAM3CSK4 has been shown to associate with African ancestry. These findings may be relevant to PCa, as prior H. pylori infection, which activates TLR1/2 signaling, is associated with PCa and occurs more frequently in AA patients. In addition, endogenous agonists of TLR1/2 have been proposed that may inflame tumors in cancer patients. Thus, both genetic differences in innate immune signaling as well as differential exposure to bacterial infection may contribute to the inflamed tumor microenvironment observed in AA PCa patients and/or explain more favorable survival of AA PCa patients after Provenge.

Mechanistically, Provenge is proposed to mediate antitumor immune responses as the primary mechanism of action, the efficacy of which is anticipated to depend upon the inflammatory nature of the vaccine, which provides antigen presentation and co-stimulation to engender antitumor immunity, as well as, potentially, the status of the tumor microenvironment, which influences the ability of antitumor immunity to infiltrate and function. Given this mechanism and in light of aforementioned racial associations we asked whether TLR1/2 responses may correlate with increased survival after Provenge. Herein, results that AA PCa patient PBMCs mount stronger overall inflammatory responses specifically after TLR1/2 activation, particularly with regards to type I/II/and III IFNs; and that stronger IFN-β induction after TLR1/2 stimulation is associated with longer survival after Provenge therapy, regardless of race.

### B. Results

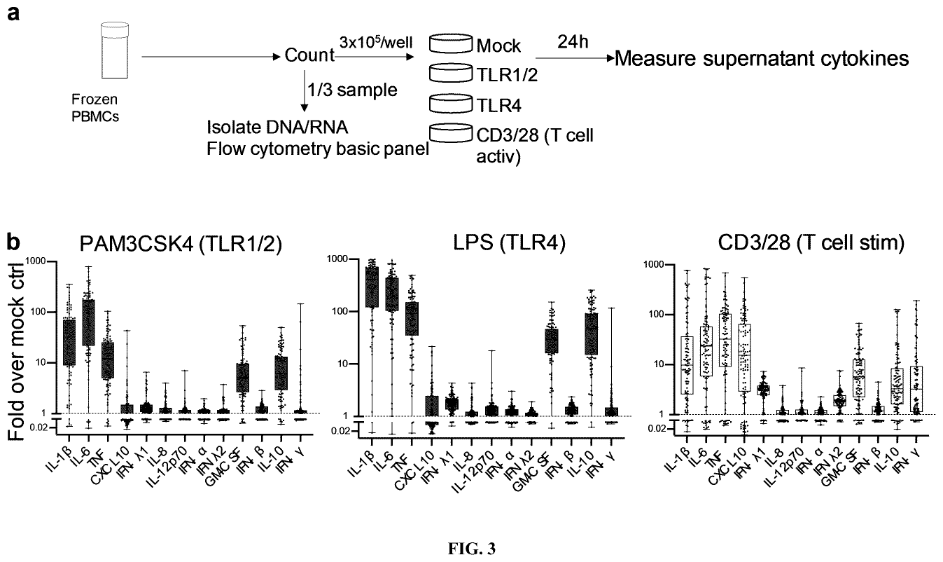

PBMCs were obtained from patients prior to initiating Provenge therapy (n=15 AA, n=91 CA). We challenged PBMCs (3×10⁵ cells) with agonists to TLR4 (LPS), TLR1/2 (PAM3CSK4), and T cells (CD3 and CD28 ligation) for 24 hours. Fold cytokine induction (relative to mock) was determined using the Biolegend Antiviral Legendplex assay, per manufacturer's instructions (FIG. 3).

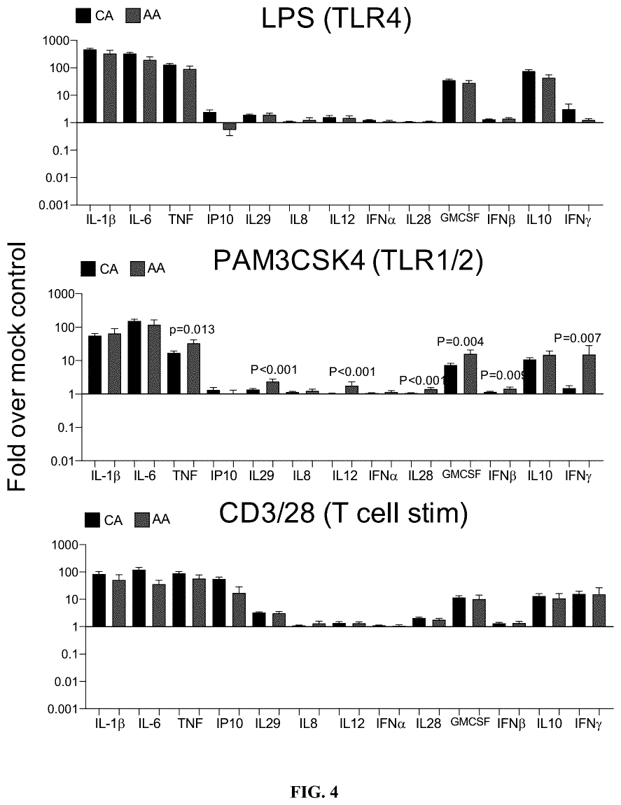

Prior reports indicate that African ancestry associates with stronger innate inflammatory responses to bacteria and TLR1/2 signaling. Inflammatory responses were compared by race. Strikingly, in line with prior reports, Black/AA PCa patients selectively had stronger induction of several cytokines relative to Caucasians, particularly interferons (IL29, IL28, IFN-β, IFN-γ) after TLR1/2. These differences were only observed in the context of TLR1/2 signaling (FIG. 4).

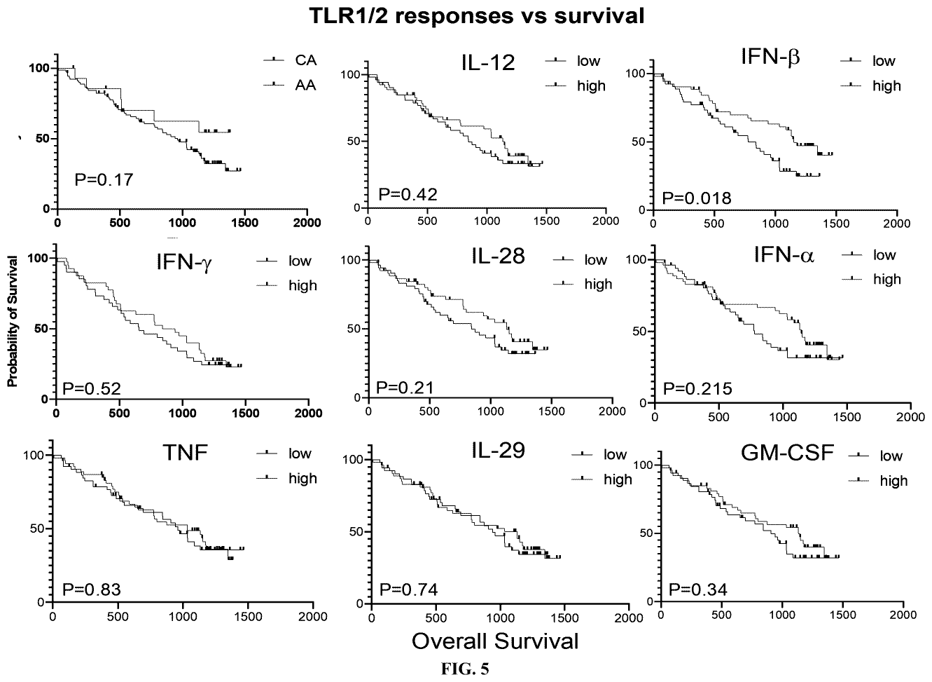

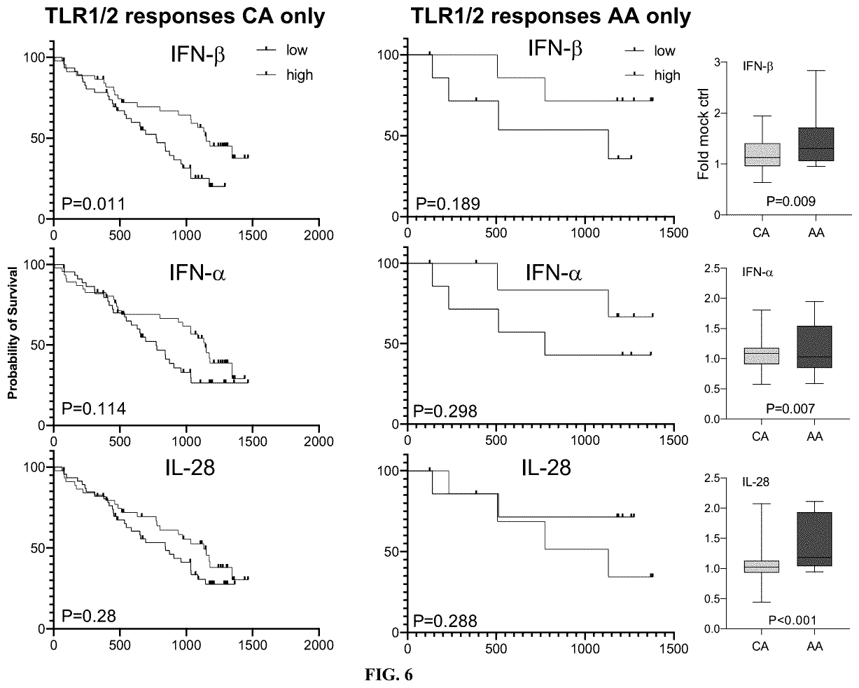

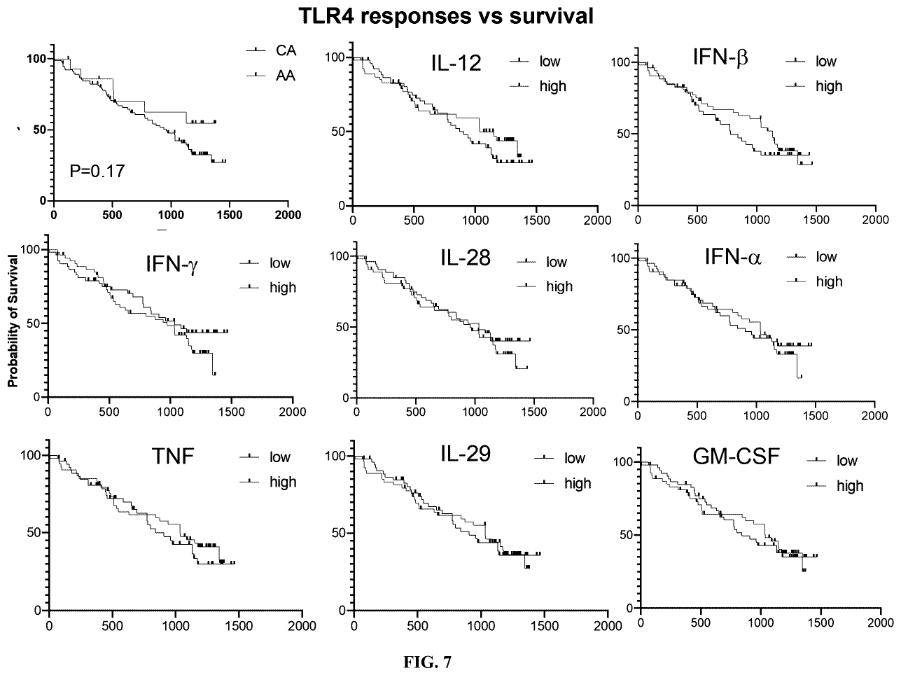

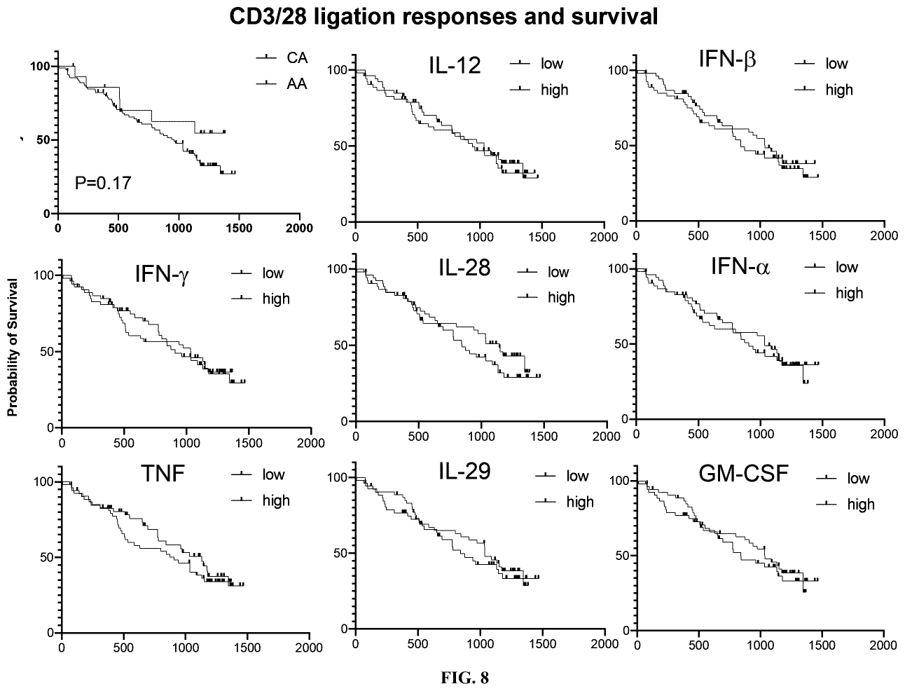

Next, it was addressed as to whether responsiveness to in vitro PRR agonist challenge associated with survival after Provenge. To accomplish this, patient survival was stratified by median induction of each cytokine indicated. Within this cohort, AA patients survived non-significantly longer than CA patients (FIG. 5). Strikingly, patients with >median (“high”) IFN-β induction survived significantly (p=0.0018) longer that patients with <median (“low”; FIG. 5). Moreover, this difference was independent of race: patients inducing stronger IFN-β responses after TLR1/2 stimulation survived longer regardless of race (FIG. 6). Importantly, stratifying patients by median cytokine responses after TLR4 agonist (FIG. 7) and T cell stimulation (FIG. 8) treatments did not reveal significant survival differences, indicating this is specific, or at least most accurately predicted by, TLR1/2 responsiveness.

3. Pretreatment Tumor and Peripheral Innate Inflammation Associates with Survival in Recurrent GBM after Polio Virotherapy.

### INTRODUCTION

It was previously discovered that recurrent GBM (rGBM) patients harboring low tumor mutation burden (TMB) with a short time to recurrence from standard of care therapy prior to treatment survived longer after polio virotherapy in a phase I clinical trial. Neither low TMB or a short time to recurrence was associated with survival in immunotherapy naïve rGBM patients. Low TMB also associated with enhanced inflammatory gene expression signatures in recurrent GBM (rGBM), but not newly diagnosed GBM tumors, possibly indicating that immune activity in the tumor may explain these associations.

Preclinical studies indicate that PVSRIPO-mediated activation of innate and adaptive antitumor immunity explains its therapeutic efficacy in manner dependent upon infection of nonmalignant tumor microenvironment constituents. Thus, the findings by the inventors that patients with high TMB are non-responsive to PVSRIPO therapy and have lower immune activity within their tumors, imply that such patients may be incapable of inducing a sufficient innate and/or adaptive immune activation in response to viral infection of the tumor. Importantly, beyond tumor-localized immune dysfunction, peripheral immune dysfunction has also been described in GBM.

### Results

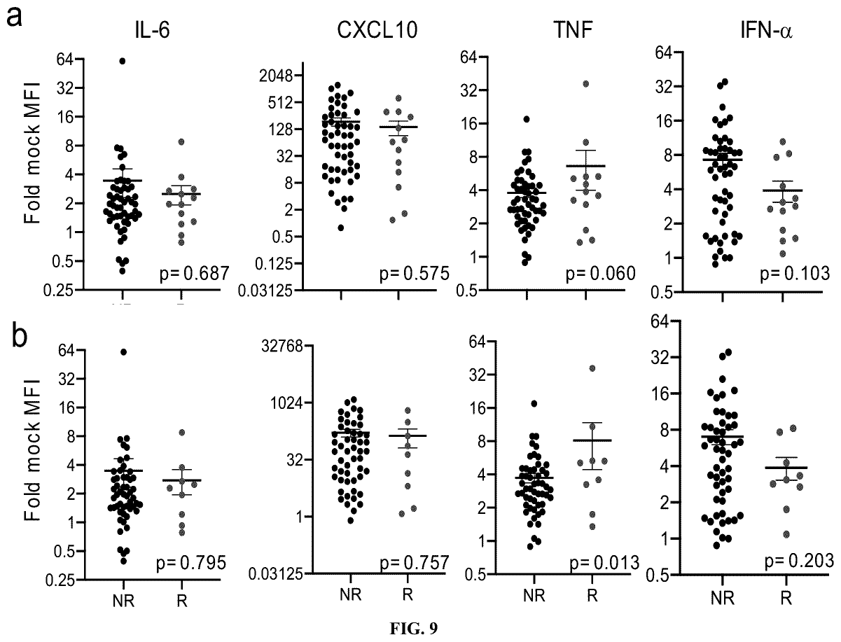

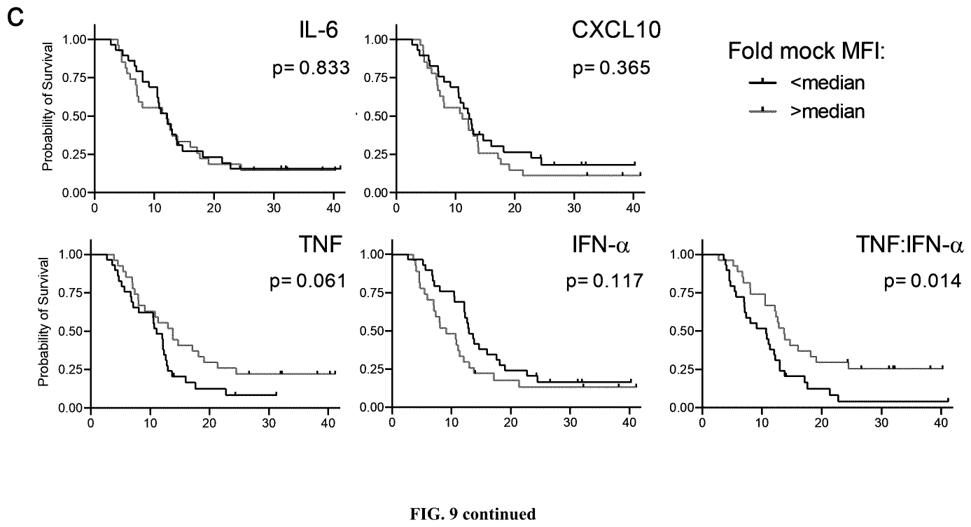

Distinct innate inflammatory responses in PBMCs to in vitro challenge with PVSRIPO identify patients surviving longer after clinical PVSRIPO therapy. It was previously reported that longer survival after PVSRIPO therapy was associated with low TMB. Recurrent GBM tumors bearing low TMB had higher tumor-intrinsic inflammatory gene expression signatures and evidence of neoantigen depletion, relative to patients with higher TMB. Based upon these findings, the inventors hypothesized that patients responding to PVSRIPO may have an ongoing, active immunological process (i.e. immune surveillance) within their tumors. In addition, the preclinical research demonstrates that oncolysis-independent activation of innate immunity within tumor associated myeloid cells primarily mediates the antitumor efficacy of PVSRIPO. Given widespread suppressed peripheral immune function in recurrent GBM patients, particularly after immunosuppressive Temozolomide, the inventors sought to determine if peripheral blood mononuclear cell (PBMC) inflammatory cytokine responses to in vitro PVSRIPO challenge, which occur largely in a CD14+ monocyte dependent manner, correlates with survival after PVSRIPO. To this end PBMCs (5×10⁵) cells per well (24-well plate) were challenged with mock or PVSRIPO (MOI 10; 5×10⁶) in vitro for 24 hours (n=56 patients), followed by multiplex cytokine secretion analysis. Cytokines induced an average of >2-fold after PVSRIPO challenge are shown (FIG. 9). It was observed that patients surviving >18 (FIG. 9a) or 24 (FIG. 9b) months after PVSRIPO therapy had lower IFN-α, but higher TNF secretion after in vitro challenge with PVSRIPO; differences in CXCL10 and IL-6 induction was not observed. Stratifying patients by median cytokine induction after in vitro PBMC challenge with PVSRIPO gave similar results: patients with higher overall TNF secretion, but lower overall IFN-secretion survived longer (FIG. 9c); stratification by induction of CXCL10 and IL-6 revealed minimal difference in survival. Patients with >median ratio of TNF:IFN-α induction live significantly (p=0.014) longer after PVSRIPO therapy. Together these findings reveal differences in the periphery of recurrent GBM patients that associate with longer survival after PVSRIPO therapy.

Prior to enrollment in the study, polio neutralizing antibody titers in patients were determined. It was asked whether there were baseline differences in anti-polio titers between patients surviving longer after PVSRIPO. Patients surviving >18 months had significantly higher anti-polio titers prior to treatment in both the phase I clinical trial cohort (FIG. 10a), and the phase II clinical trial cohort (FIG. 10b). This difference in baseline anti-polio titers may either represent overall higher intact immunological memory in patients surviving longer, or may represent a contribution of polio memory to eventual therapy outcome.

In assessing poliovirus titers, the method of E. J. BOONE & p ALBRECHT, Office of Biologics, National Center for Drugs and Biologics, FDA (‘Conventional and Enhanced Plaque Neutralization Assay for Polio Antibody’) J. Vir. Methods 6 (1983) 193-202 was used. To this end, serum samples were thawed at room temperature and processed as follows:
1. Serial dilution of the samples is performed in DMEM (2% fetal bovine serum; 10,000 U penicillin/ml, 10,000 μg streptomycin/ml, 25 μg amphothericin B/ml) at 1:4; 1:16, 1:64, 1:256, 1:1024 and 1:4096 in sterile, disposable Eppendorf tubes. To achieve these dilution factors, 100 μl of the original serum sample is pipetted into 300 μl of DMEM followed by serial dilution in 4-fold increments. All samples contain ˜50 plaque forming units (PFU) of glp-produced PVSRIPO. The procedure takes place in a biosafety cabinet. For the few samples where the material available was less than 100 μl, the dilution factors were adjusted downward to 1:16, 1:64, etc.
2. The serum:DMEM:virus mixture was incubated in sealed Eppendorf tubes o/n at 20° C. in a rotating device.
3. The diluted samples (300 μl) were applied to Hela R19 cells grown o/n in sterile, disposable 6-well plates.
4. The SOP for plaque assay (step 3 onward) applies.
5. Stained plates are counted to determine plaque numbers for each diluted sample.
6. The neutralizing antibody titer was determined as the serum dilution that achieved 50% of plaque neutralization.

### Materials

Dulbecco's minimal essential medium (DMEM), sterile. Invitrogen, cat #11995
Fetal bovine serum (FBS), sterile. Invitrogen, cat #16000, lot #505985
Antibiotic-antimicotic, sterile. Invitrogen, cat #15240
Eppendorf tube. Autoclaved. Axygen, cat #MCT-150-C
Cellstar 6-well plates, sterile. Greiner, cat #657160

### PVS-RIPO

PVSRIPO is a recombinant rhinovirus/poliovirus (PV) chimera administered by intratumoral injection, that is being developed to treat patients with solid tumor cancers. It is a modified version of the serotype 1 live-attenuated (Sabin™) PV vaccine (PVIS) with its cognate internal ribosome entry site (IRES) replaced with that of human rhinovirus type 2 (HRV2). Its immunogenic properties and low potential for long-term sequelae are expected to be similar to the vaccine. PVIS has been safely administered to >10 billion individuals worldwide without untoward long-term sequelae. The administration of PVIS in humans leads to neutralizing immunity to PV. The foreign IRES of PVSRIPO causes neuronal incompetence: a failure to recruit host ribosomes, translate viral genomes, and propagate in neurons, which ablates neurovirulence (ie, PVSRIPO does not cause polio-related neurologic sequelae) (Dobrikova 2012).

The utility of PVSRIPO in treating cancer is tied to the (1) expression of CD155 (the PV receptor [Mendelsohn 1989]) in specific cell types, and the (2) effect of the foreign IRES on viral replication. Because cluster of differentiation (CD)155 is expressed on malignant cells from virtually all solid tumors (Takai 2008; Chandramohan 2017; Liu 2019; Masson 2001; Bevelacqua 2012; Carlsten 2009; Nishiwada 2015; Sun 2020; Zhang 2020), and many myeloid components of the tumor stroma (invading monocytes, tumor associated macrophages, dendritic cells [DCs]) (Freistadt 1993), PVSRIPO has a tropism for the major components of the tumor and tumor microenvironment (TME). And while the presence of CD155 is sufficient for PVSRIPO entry into a cell, it is not sufficient for PVSRIPO replication. For example, while CD155 is expressed on spinal cord/medullary motor neurons, the presence of a foreign, neuro-incompetent IRES precludes PVSRIPO replication and any potential for neurovirulence. In malignant cells, viral replication (and subsequent cytotoxicity) is due to constitutive activation of the protein kinase C (PKC)-RAF-extracellular signal-regulated kinase (ERK)1/2-mitogen-activated protein kinase-interacting serine/threonine-protein kinase (MNK)1/2 pathway which upregulates the protein synthesis machinery and allows unfettered translation of the PVSRIPO genome. In normal cells, which do not proliferate uncontrollably, this PKC-RAF-ERK1/2-MK1/2 pathway is not constitutively activated, so PVSRIPO replication is hindered. In addition, while most viruses infect antigen presenting cells (APCs)/DCs and suppress antigen presentation to enable immune evasion, infection of APCs/DCs by PVSRIPO results in marginal viral replication, enhancement of APC/DC-mediated antigen presentation, and interferon (IFN)-dominant inflammation in the TME (Brown 2017). The enhancement of APC/DC activity by PVSRIPO may also be related to the effects of the virus on CD155 expression, which is down regulated in cells infected with PVSRIPO (Mosaheb 2020). Since CD155 is the ligand for T cell immunoreceptor with immunoglobin (Ig) and immunoreceptor tyrosine-based inhibitor motif (ITIM) domains (TIGIT) which is an emerging immune checkpoint thought to play a central role in limiting anti-tumor immune responses, down regulation of CD155 expression is predicted to reduce TIGIT-induced immunosuppression (Harjunpää 2020).

### 4. Baseline Innate Immune Function and Response to PVSRIPO in Melanoma Patients

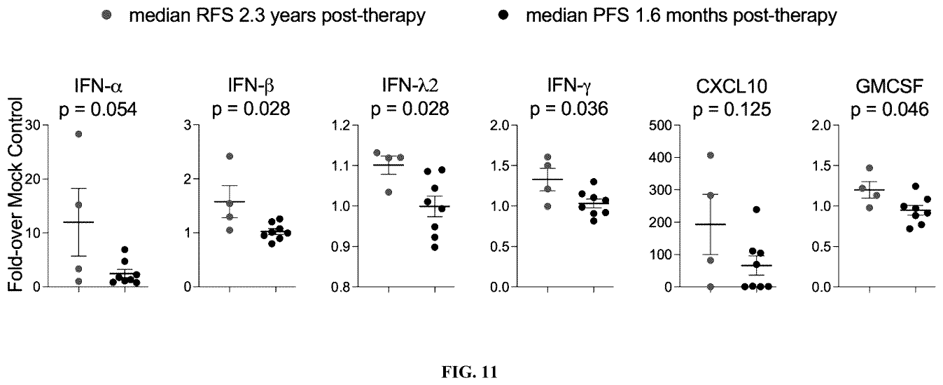

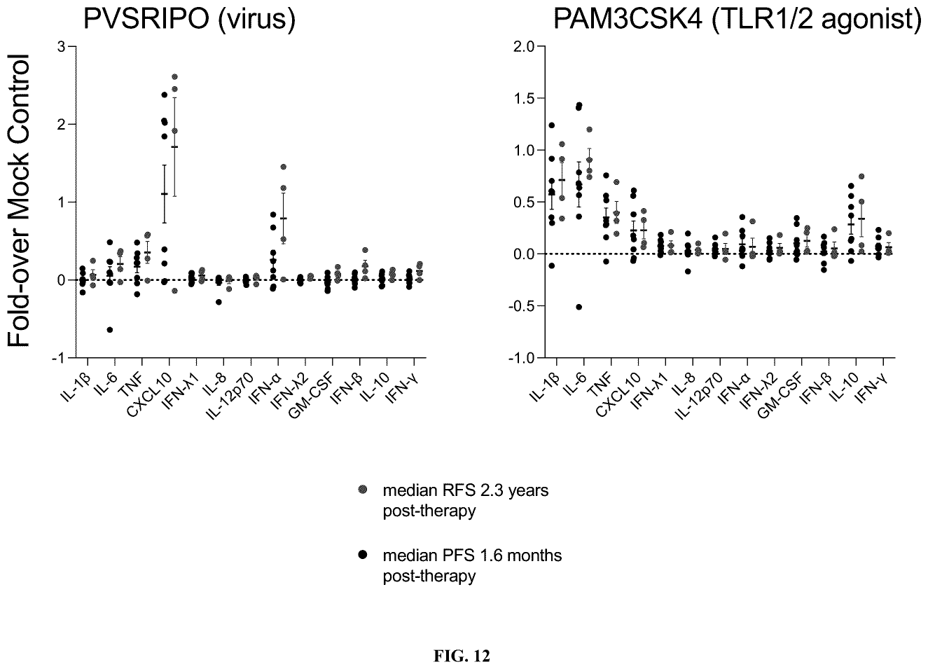

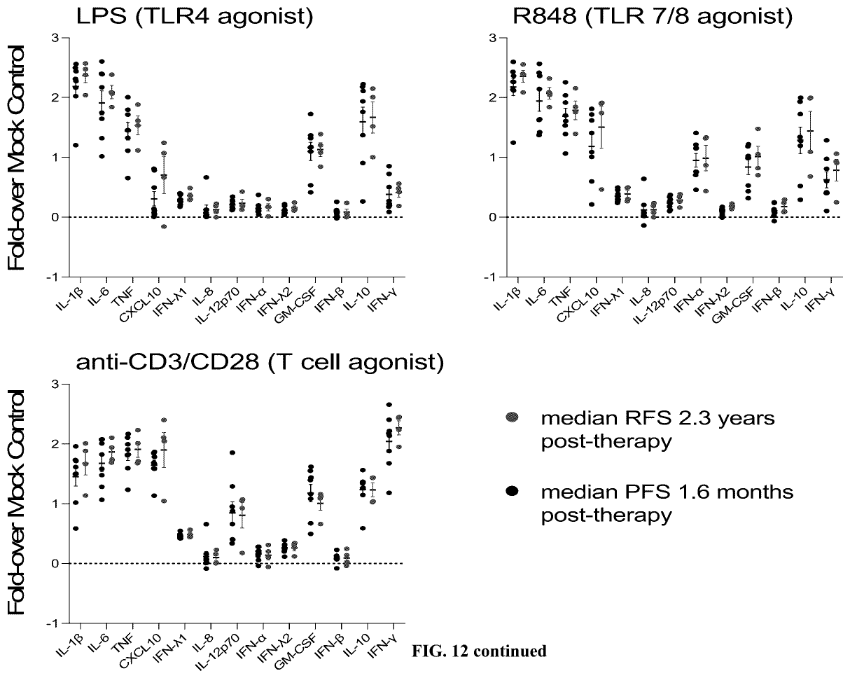

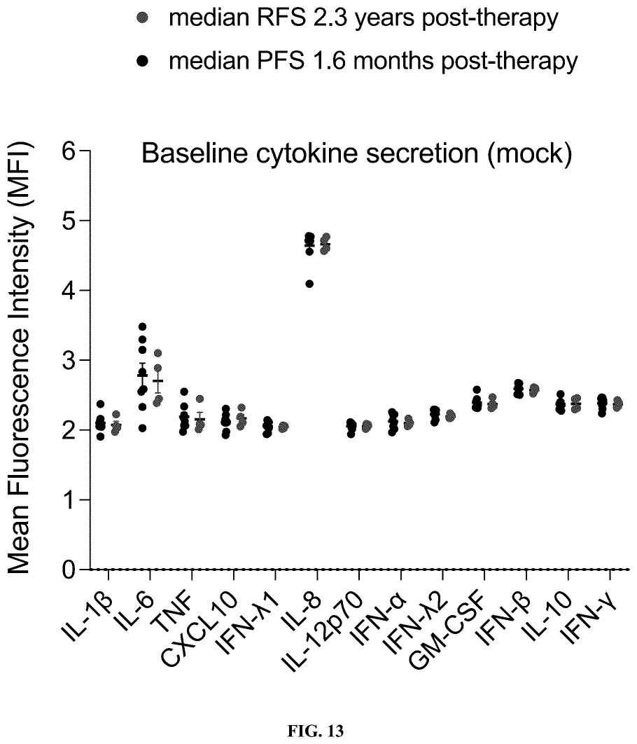

Since innate inflammation induced in myeloid cells mediates antitumor efficacy of PVSRIPO (lerapolturev) in mice^{1,2}, we hypothesized that peripheral capacity to respond to PVSRIPO prior to therapy may associate with therapy outcome. We developed a peripheral immune function assay that measures pro-inflammatory cytokine responses of pre-treatment PBMCs after in vitro challenge with lerapolturev or other stimuli (FIGS. 11-13). The fold increase in cytokine production after 24 hours of stimulation with lerapolturev over mock treatment is shown (FIGS. 11 and 12). Patients with median RFS (recurrence free survival) of 2.3 years after lerapolturev (Patients 8, 9, 11, 12) including the 3 patients with known pre-lerapolturev inflamed TME who received anti-PD-1 therapy within 30 days of lerapolturev (8, 9 and 12), had significantly higher production of IFN-β, IFN-λ2, IFN-γ, and GMCSF compared to 8 patients with median PFS (progression free survival) 1.6 months after lerapolturev. In addition, CXCL10 production was higher after LPS and CD3/28 treatment in the 4 patients with RFS 2.3 years after lerapolturev compared to 8 patients with median PFS 1.6 months (FIG. 12). No difference in baseline cytokine secretion (mock treatment) was observed between patients with RFS 2.3 years vs PFS 1.6 months (FIG. 13), indicating the aforementioned superior post-stimulation cytokine levels in patients with RFS 2.3 years were due to differential sensitivity to in vitro stimulation. Peripheral pre-treatment immune responsiveness (FIG. 11) observed in patients with RFS 2.3 years, imply that an active and/or proficient pre-treatment immune status in patients may result in favorable clinical outcomes.

### REFERENCES

1. Brown M C, Mosaheb M M, Mohme M, Mckay Z P, Holl E K, Kastan J P, Yang Y, Beasley G M, Hwang E S, Ashley D M, Bigner D D, Nair S K, Gromeier M. Viral infection of cells within the tumor microenvironment mediates antitumor immunotherapy via selective TBK1-IRF3 signaling. Nat Commun. 12(1): 1858, 2021.
2. Brown M C, Holl E K, Boczkowski D, Dobrikova E, Mosaheb M, Chandramohan V, Bigner D D, Gromeier M, Nair S K. Cancer immunotherapy with recombinant poliovirus induces IFN-dominant activation of dendritic cells and tumor antigen-specific CTLs. Sci Transl Med. 9(408), 2017. PMCID: PMCPMC6034685.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method comprising:
(a) contacting a patient sample with an innate immune agonist; and
(b) measuring the level of at least one inflammatory cytokine,
wherein the level of inflammatory cytokine production is indicative of responsiveness of the patient to an immunotherapy, wherein the patient has cancer and the immunotherapy comprises an anti-cancer immunotherapeutic.

2. The method of claim 1, further comprising administering an immunotherapy to the patient when the level of inflammatory cytokine production is at or above a reference level.

3. (canceled)

4. The method of claim 2, wherein the immunotherapy comprises an agent selected from an immune checkpoint inhibitor, vaccine, adjuvant, cytokine, human cell therapy, microorganism, and virus.

5. (canceled)

6. The method of claim 2, wherein the immunotherapy comprises an oncolytic virus selected from an oncolytic poliovirus, adenovirus, HSV-1 virus, reovirus, poxvirus, Newcastle Disease virus, measles virus, Seneca Valley virus, hemagglutinating virus of Japan Envelope (HVJ-E) virus, herpes virus, parvovirus, retrovirus, PVS-RIPO, paleorep, GEN0101, seprehvir talimogene laherparepvec, adenovirus VCN-01, adenovirus ICORVIR-5, HF10, GL-ONC1, DNX-2401, enadenotucirev, and a derivative of any of the aforementioned.

7. The method claim 1, wherein the inflammatory cytokine is selected from: TNFα, IL-12, IFN-α, IFN-β, IFN-γ, IFN-λ2, IL-28, IL-29, CXCL9, CXCL10, GMCSF, IL-1β, IL-6, IFN-γ1, and IL-8.

8. The method of claim 1, wherein the innate immune agonist is selected from the group consisting of a pattern recognition receptor (PRR) agonist, a microorganism or antigen thereof, a virus or antigen thereof, a STING/TMEM173 agonist and a T cell agonist.

9. (canceled)

10. The method of claim 8, wherein the pattern recognition receptor agonist is selected from a toll-like receptor agonist, C-type lectin receptor agonist, NOD-like receptor agonist, and RIG-I like receptor (RLR) agonist.

11.-12. (canceled)

13. The method claim 1, wherein the measuring step comprises measuring the level of at least two inflammatory cytokines.

14. (canceled)

15. The method claim 1, wherein the level of inflammatory cytokine production is increased and the increased inflammatory cytokine production is indicative of immune proficiency to respond to a cancer immunotherapy.

16. (canceled)

17. The method of claim 1, further comprising administering an oncolytic virus to the patient.

18. The method of claim 2, further comprising administering an oncolytic virus to the patient.

19.-20. (canceled)

21. The method of claim 1, wherein the patient has melanoma cancer, wherein the innate immune agonist is a TLR4 agonist (LPS) or +-RNA virus, and wherein the inflammatory cytokine is TNFα.

22. The method of claim 1, wherein the patient has pancreatic cancer, wherein the innate immune agonist is a viral antigen and the inflammatory cytokine is TNFα or CXCL10, or wherein the innate immune agonist is a TLR1/2 agonist (PAM3CSK4) and the inflammatory cytokine is IFN-β.

23. (canceled)

24. The method of claim 1, wherein the patient has glioblastoma cancer, wherein the innate immune agonist is a polio viral antigen, and wherein the inflammatory cytokine is TNFα.

25. A method comprising:
(a) obtaining a serum sample from a subject diagnosed with a cancer;
(b) detecting the level of antibody specific to a viral antigen in the serum sample;
(c) comparing the level of neutralizing antibodies in the sample to a reference level; and
(d) treating the subject with an oncolytic viral therapy when the level of the neutralizing antibody in the sample is above the reference level.

26. (canceled)

27. The method of claim 25, further comprising before the treating step (d), the step of administering to the subject a booster virus related to the virus of the oncolytic viral therapy.

28. The method of claim 25, further comprising before the obtaining step (a), the step of administering an oncolytic viral therapy.

29. (canceled)

30. The method of claim 25, wherein the subject has a solid tumor, and a level of the antibody in the serum sample above the reference level is indicative of immune proficiency of the solid tumor to respond to the oncolytic viral therapy.

31.-32. (canceled)

33. The method of claim 25, wherein the cancer comprises a cancer cell expressing CD155 and the oncolytic viral therapy comprises a polio virus or derivative thereof.

34. (canceled)

35. The method of claim 25, further comprising treating the subject with at least one anti-cancer immunotherapy.

36.-38. (canceled)
